(19) United States
(12) Patent Application Publication
(10) Pub. No.: US 2026/0279513 A1
(43) Pub. Date: Sep. 17, 2026

(54) PREDICTION OF THE UV PERFORMANCE OF SUNSCREENS

(71) Applicant: BASF SE, Ludwigshafen am Rhein (DE)

(72) Inventors: Myriam SOHN, Grenzach-Wyhlen (DE); Bernd HERZOG, Grenzach-Wyhlen (DE)

(21) Appl. No.: 18/871,963

(22) PCT Filed: Jun. 9, 2023
(86) PCT No.: PCT/EP2023/065533

§ 371 Date: Dec. 5, 2024

(30) Foreign Application Priority Data
Jun. 10, 2022 (EP) 22178378.0

(51) Int. Cl. G16C 60/00

(52) U.S. Cl. CPC G16C 60/00

(57) ABSTRACT

The present invention relates to sunscreen filter composition development. In order to accurately determine a sunscreen filter composition with a formulation type, a computer-implemented method (200) is provided for determining a performance of a sunscreen product comprising at least one UV filter, the method comprising the steps of: a) providing (210) a computational model to calculate a performance index of a sunscreen product, wherein the computational model applies a continuous film thickness distribution defined by a gamma distribution as a model to determine a film thickness distribution of the applied sunscreen product; b) providing (220) a desired formulation type of the sunscreen product; c) adapting (230) the shape parameter c of the gamma distribution to the desired formulation type; d) determining (240) the performance index of a UV-filter composition for the desired formulation type utilizing the adapted shape parameter of the gamma distribution; and e) providing (250) the determined performance index of the sunscreen product having the desired formulation type, preferably usable for manufacturing the sunscreen product, wherein the shape parameter c is a function of a total filter concentration, TFC, wherein the function is a saturation like function with a larger slope at a lower TFC and a smaller slope at a higher TFC, and an amplitude of the function is related to a performance of a formulation type in terms of the performance index of the sunscreen product.

### FIELD OF THE INVENTION

The present invention relates to a computer-implemented method and an apparatus for determining a performance of a sunscreen product comprising at least one UV filter, to a method and an apparatus for manufacturing a sunscreen product, to a method and an apparatus for validating manufacture of a sunscreen product, to a computer program element, and to a computer-readable medium.

### BACKGROUND OF THE INVENTION

Sunscreen filter compositions are prepared from a palette of different UV-filter substances, which are known for their different UV absorbing characters. When creating a sunscreen filter composition the designer selects substances and combines them to achieve a desired sun protection performance.

Today sunscreen developers have computational tools available, for example the sunscreen simulator of BASF (https://sunscreensimulator.basf.com/Sunscreen_Simulator/login), which can predict the sun protection performance of a UV-filter combination. Currently, the calculation of the sun protection performance is based on the filter combination only. For example, a computational method for calculation of the UV screening performance is available in B. Herzog and U. Osterwalder, Pure and Applied Chemistry. 87, 937 (2015). However, the vehicle in which the filters are use may impact the sun protection performance, different formulations with the same UV-filter composition may therefore exhibit different sun protection performances.

Therefore, the current computational tools may have difficulty in evaluating accurately the sun protection performance of a particular formulation type (e.g., oil-in-water fluid, oil-in-water cream, etc.). Thus, extensive in vivo testing has to be carried out.

### SUMMARY OF THE INVENTION

There may be a need to accurately determine the sun protection performance of a sunscreen filter composition in a specific formulation type.

The object of the present invention is solved by the subject-matter of the independent claims, wherein further embodiments are incorporated in the dependent claims. It should be noted that the following described aspects of the invention apply also for the computer-implemented method and the apparatus for determining a performance of a sunscreen product comprising at least one UV filter, the method and the apparatus for manufacturing a sunscreen product, the method and the apparatus for validating manufacture of a sunscreen product, the computer program element, and the computer-readable medium.

According to a first aspect of the present invention, there is provided a computer-implemented method for determining a performance of a sunscreen product comprising at least one UV filter, the method comprising the steps of:
a) providing (210) a computational model to calculate a performance index of a sunscreen product, wherein the computational model applies a continuous film thickness distribution defined by a gamma distribution as a model to determine a film thickness distribution of the applied sunscreen product;
b) providing (220) a desired formulation type of the sunscreen product;
c) adapting (230) the shape parameter c of the gamma distribution to the desired formulation type;
d) determining (240) the performance index of a UV-filter composition for the desired formulation type utilizing the adapted shape parameter of the gamma distribution; and
e) providing (250) the determined performance index of the sunscreen product having the desired formulation type, preferably usable for manufacturing the sunscreen product,
   wherein the shape parameter c is a function of a total filter concentration (TFC) wherein the function is a saturation like function with a larger slope at a lower TFC and a smaller slope at a higher TFC, and an amplitude of the function is related to a performance of a formulation type in terms of the performance index of the sunscreen product.

The calculation of the sun protection performance is currently determined based on the properties of the UV filter(s) (e.g., extinction, photostability alone, or photostability of a combination thereof) and a model to describe the layer of applied sunscreen, since the UV transmission depends on the thickness and thickness distribution of the applied layer of sunscreen. The gamma distribution model is an asymmetrical distribution suited to describe the profile of the sunscreen applied films, it contains a shape parameter that is adjustable so that the calculated sun protection performance of a series of test sunscreens matches as best as possible a reference sun protection performance which can be for example the sun protection factor (SPF) in vivo (ISO24444).

The computer-implemented method and the apparatus as described herein adapt the shape parameter c in the model, which describes the layer of applied sunscreen for different formulation types (also called formulation vehicles) taken as reference, so that the determined sun protection performance considers also the formulation type or formulation vehicle (e.g., oil-in-water fluid, oil-in-water cream, etc.) in addition to the properties of UV filters.

The shape parameter c is a saturation like function of the TFC. If the at least one UV-filter composition comprises one UV filter, the TFC can be expressed as the concentration of the UV filter in percent (weight/volume). If the at least one UV-filter composition comprises two or more UV filters, each UV filter having a respective concentration in percent, the TFC is the sum of the concentrations in percent of the two or more UV filters.

Examples of the saturation like function will be described hereinafter. An exemplary saturation like function is shown in FIG. 5A. As will be explained in detail hereinafter and in particular with respect to FIGS. 5A and 5B, the amplitude of the function is related to a performance of a formulation type (or formulation vehicle) in terms of the performance index of the sunscreen product. By adjusting only this amplitude, the problem of an intersection of the curves of the shape parameters when plotted against the total filter concentration for different formulation types (or formulation vehicles) can be overcome.

Having the possibility of selecting the formulation type, the desired solution may be tailored to the user's needs. The desired solution may also reduce the difference between the calculated sun protection performance and the measured sun protection performance in vivo, thereby reducing extensive in vivo testing's at the user level.

This will be explained in detail hereinafter and in particular with respect to the example shown in FIG. 3.

Additionally, the determined calculated performance index of the sunscreen product may be used for controlling or validating the manufacture of a sunscreen product, and may thus reduce extensive trial runs in manufacturing a desired sunscreen product. This will be discussed in detail hereinafter and in particular with respect to the examples shown in FIGS. 7 to 10. According to an embodiment of the present invention, the performance index includes sun protection factor (SPF) and/or UVA protection factor (UVA-PF).

According to an embodiment of the present invention, the saturation like function comprises:

$$c = a 1 + a 2 \cdot ( \frac{{TFC}^{a 3}}{a 4 + {TFC}^{a 3}} )$$

where c is the shape parameter, a1, a2, a3, and a4 are adjustable parameters, which are determined for each formulation type.

We observed that certain formulation types always display a higher SPF in vivo than others while containing the same UV-filter composition. To factor in the different behaviour of different formulation types, the shape parameter c was determined for each formulation type. The shape parameter c may comprise a specific set a1, a2, a3 and a4 of adjustable parameters for each formulation type. Those parameters determine the form of the curve when plotting the shape parameter against the TFC. The higher the shape parameter, the higher the calculated SPF. The calculated SPF may also be referred to as simulated SPF or estimated SPF.

According to an embodiment of the present invention, the adjustable parameters, a1, a2, a3, and a4 may have the following ranges:

$$0.35 \leq a 1 \leq 0.6 ;$$

$$0.3 \leq a 2 \leq 0 \text{.68} ;$$

$$0.9 \leq a 3 \leq 1.5 ; and$$

$$a 4 \leq 8 .$$

As will be explained in detail hereinafter and in particular with respect to Tables 1 to 3, an improved accuracy for determining the performance index can be achieved with adjustable parameters within the above-described preferred ranges.

According to an embodiment of the present invention, the saturation like function comprises:

$$c = a 1 + a 2 \cdot ( \frac{{TFC}^{a 3}}{a4 + {TFC}^{a 3}} ) + \frac{a 5}{4} - a 5 \cdot {( REAE - 0.5 )}^{2}$$

where c is the shape parameter, a1, a2, a3, a4, and a5 are adjustable parameters, which are determined for each formulation type.

In this example, the saturation like function further comprises a parameter a5, which is only needed when the filter composition contains at the same time UV absorbers in the oil and in the water phase, where REAE means the relative erythemally active extinction in the oil-phase and relates to the increase of the performance index that can be obtained when filters are present at the same time in the oil and in the water phase of an emulsion (B. Herzog and U. Osterwalder, Pure and Applied Chemistry. 87, 937 (2015)). Parameter a5 can vary between 0 and 1. These parameters are determined for each formulation type. If the filter systems of the formulations do contain only UV filters in the water phase or only UV filters in the oil phase, the parameter a5 may be neglected.

According to an embodiment of the present invention, the parameters, a1, a2, a3, a4, and a5 may have the following ranges:

$$0.35 \leq a 1 \leq 0.6 ;$$

$$0.3 \leq a 2 \leq 0 \text{.68} ;$$

$$0.9 \leq a 3 \leq 1.5 ;$$

$$a 4 \leq 8 ; and$$

$$0 \leq a 5 \leq 1 .$$

According to an embodiment of the present invention, the saturation like function comprises:

$$c = a 1 + a 2 \cdot ( 1 - {e}^{( - TFC \cdot a 6 )} )$$

where c is the shape parameter, a1, a2, and a6 are adjustable parameters, which are determined for each formulation type, and the parameters a1 and a2 are in the same ranges as described above, and 0.1≤a6≤0.9.

According to an embodiment of the present invention, the saturation like function comprises:

$$c = a 1 + a 2 \cdot ( 1 - {e}^{( - TFC \cdot a 6 )} ) + \frac{a 5}{4} - a5 \cdot {( REAE - 0.5 )}^{2}$$

where c is the shape parameter, a1, a2, and a6 are adjustable parameters in the same ranges as described in the above embodiment, and 0≤a5≤1.

According to an embodiment of the present invention, the performance index includes sun protection factor (SPF) and/or UVA protection factor (UVA-PF).

In an example, the formulation type comprises a formulation with water or a formulation without water.

According to an embodiment of the present invention, the formulation type (or formulation vehicle) comprises one or more of: oil-in-water fluid emulsion, oil-in-water cream emulsion, water-in-oil emulsion, oil-in-water-in-oil emulsion, water-in-oil-in-water emulsion, water-in-silicone emulsion, silicone-in-water emulsion, polymeric gel cream, lipophilic monophase oil, lipophilic monophase gel, lipophilic monophase stick, lipophilic-alcoholic mixture, hydrophilic monophase fluid, hydrophilic monophase gel, and powder.

The formulation may be a spray, a cream, a lotion, a mousse (foam), and a powder, etc. and might be packed accordingly in a bottle, a jar, a pump spray, or an aerosol with an appropriate applicator.

Hydrophilic refers to water and compounds soluble or miscible in water.

FIGS. 4A-4C illustrate an exemplary implementation of the interactive user interface that enable an evaluation of the interaction between the formulation type and the performance index.

According to an embodiment of the present invention, the parameters of the function determining the adapted shape parameters is derived from experimental data of SPF and/or UVA-PF.

The experimental data of SPF and/or UVA-PF may be in vivo and/or in vitro.

According to an embodiment of the present invention, the sunscreen product further comprises one or more cosmetic ingredients.

Examples of the cosmetic ingredients may include, but are not limited to, emulsifiers, emulsifying polymers, emollients, hydrophilic humectants, waxes, thickening polymers, consistency enhancers, silicon based compounds, pH regulators, actives, preservatives, and perfumes.

According to a second aspect of the present invention, there is provided a method for manufacturing a sunscreen product, the method comprising the steps of:
providing a composition comprising one or more UV-filter substances with a formulation type;
determining a calculated performance index of a desired sunscreen product according to the method of the first aspect and any associated example; and
manufacturing a sunscreen product using the composition comprising one or more UV filter substances within the formulation type.

This will be explained in detail hereinafter and in particular with respect to the example shown in FIG. 7.

According to an embodiment of the present invention, the method further comprises:
providing a measured performance index of the manufactured sunscreen product; and
comparing the measured performance index of the manufactured sunscreen product with the calculated performance index of the desired sunscreen product to determine if the manufactured sunscreen product fulfils predetermined quality criteria.

According to an embodiment of the present invention, the method further comprises:
in response to determining that the manufactured sunscreen product fulfils the predetermined quality criteria, generating a control signal usable for controlling a production process; or
in response to determining that the manufactured sunscreen product does not fulfil the predetermined quality criteria, generating a warning signal usable to signify invalidity of the manufactured product and/or generating an optimization signal usable to change the provided composition such that the manufactured sunscreen product fulfils the predetermined quality criteria.

This will be explained in detail hereinafter and in particular with respect to the example shown in FIG. 7.

According to third aspect of the present invention, there is provided a method for validating manufacture of a sunscreen product, the method comprising the steps of:
providing a composition comprising one or more UV-filter substances with a formulation type wherein the provided composition differs from the composition of an existing produced sunscreen product by at least one different substance and/or different formulation type;
determining a calculated performance of the provided composition according to the first aspect and any associated example;
manufacturing a sunscreen product using the composition comprising one or more UV filter substances within the formulation type wherein the manufactured composition differs from the composition of an existing produced sunscreen product by the at least one different substance and/or the different formulation type; and
comparing a measured performance index of manufactured composition and the existing performance index of the existing sunscreen product to validate the at least one substance and/or the different formulation type.

This will be explained in detail hereinafter and in particular with respect to the example shown in FIG. 8.

According to a fourth aspect of the present invention, there is provided an apparatus for determining a performance index of a composition comprising one or more UV-filter substances with a formulation type, the apparatus comprising one or more processing units configured to determine the performance index of a composition of filter substances to form the sunscreen composition, wherein the one or more processing units include instructions, which when executed on the one or more processing units execute the method steps of the first aspect and any associated example.

This will be explained in detail hereinafter and in particular with respect to the examples shown in FIGS. 1 and 2.

According to a fifth aspect of the present invention, there is provided an apparatus for manufacturing a sunscreen product. The apparatus comprises a monitoring apparatus and a dosing equipment. The monitoring apparatus is configured to control the dosing equipment to manufacture the sunscreen product.

This will be explained in detail hereinafter and in particular with respect to the examples shown in FIG. 9.

According to a sixth aspect of the present invention, there is provided an apparatus for validating manufacture of a sunscreen product, the apparatus comprising one or more processing unit(s) configured to validate production of a sunscreen product, wherein the processing unit(s) include instructions, which when executed on the one or more processing unit(s) preform the method.

This will be explained in detail hereinafter and in particular with respect to the examples shown in FIG. 10.

According to another aspect of the present invention, there is provided a computer program element comprising instructions, which when executed by a processing unit, cause the processing unit to carry out the steps of the method of the first aspect and any associated example.

According to a further aspect of the present invention, there is provided a computer-readable medium having stored thereon the computer program element.

In an embodiment, the UV-filter substance may comprise a specific compound that impedes the passage of ultraviolet light. In other words, the UV-filter substance may include soluble or insoluble, organic or inorganic agents that protect the skin from damaging effects of sunlight such as erythema by absorbing or blocking ultraviolet radiation. The soluble agents work by absorbing UV rays, the insoluble or particulate agents work by absorbing and additionally reflecting and/or scattering UV rays. Different jurisdictions may allow different UV filter substances. The UV-filters allowed in cosmetic products in the European Union are listed in the Annex VI of the Regulation “EC” No. 1223/2009 of the European Parliament and of the Council.

In an embodiment, exemplary emulsifiers used to form an oil-in-water emulsion may include one or more of:
glucose derivatives such as cetearyl glucoside, arachidyl glucoside, lauryl glucoside, coco glucoside, polyglyceryl-3 methylglucose distearate, methyl glucose sesquistearate;
sucrose derivative such as sucrose polystearate, sucrose palmitate;
sorbitol derivatives such as polysorbate derivatives;
inulin derivatives such as inulin lauryl carbamate;
glycerides of fatty acids such as glyceryl stearate, glyceryl stearate SE, glyceryl stearate citrate;
glumatic acid derivatives such as sodium stearoyl glutamate;
sulfosuccinic acid derivatives such as disodium cetearyl sulfosuccinate;
phosphoric acid derivatives such as potassium cetyl phosphate; ceteh-10 phosphate, C20-22 alkyl phosphate;
fatty acid esters of polyglyceryl such as polyglyceryl-10 stearate, polyglyceryl-6 behenate
Oxyakenylated fatty alcohol such as ceteareth-20, steareth-21, beneheth-25;
Oxyakenylated fatty acid such as PEG-100 stearate;
oxyalkenylated organomodified silicone/polysiloxane/polyalkyl/polyether copolymers and derivatives such as PEG-12 dimethicone; and
phospholipids based such as lecithin derivatives.

In an embodiment, exemplary emulsifiers used to form a water-in-oil emulsion may include one or more of:
glycerides of fatty acids such glyceryl oleate, sorbitan laurate;
sorbitan esters such as sorbitan oleate;
fatty acid esters of polyglyceryl such as polyglyceryl-3-diisostearate, polyglyceryl-2-dipolyhydroxystearate, polyglyceryl-4 isostearate;
Oxyakenylated fatty alcohol such steareth-2;
Oxyakenylated fatty acid such as PEG-30 dipolyhydroxystearate; and
organomodified silicone/polysiloxane/polyalkyl/polyether copolymers and derivatives such as cetyl dimethicone copolyol, cetyl PEG/PPG-10/1 Dimethicone, PEG-10 dimethicone.

In an embodiment, exemplary lipophilic thickeners used to increase the viscosity of an emulsion may include one or more of:
fatty alcohols such as cetyl alcohol, cetearyl alcohol, stearyl alcohol; behenyl alcohol;
fatty acids such as stearic acid; palmitic acid;
fatty acid esters such as myristyl stearate, pentaerythrityl distearate, cetyl palmitate;
Tribehenin, dextrin palmitate;
waxes such as beeswax, carnauba wax, microcrystalline wax, ceresin, ozocerite, Oryza sativa bran Wax, sunflower wax;
Hydrogenated vegetable oil, hydrogenated castor oil, Hydrogenated vegetable glycerides;
Hydrogenated Castor Oil/Sebacic Acid Copolymer
Poly C10-30 Alkyl Acrylate;
Polyamide derivatives such as polyaminde-8; and
Silica based derivatives such as silica, silica dimethyl silylate.

In an embodiment, exemplary hydrophilic stabilizers/thickeners used to increase the viscosity of an emulsion may include one or more of:
natural gums such as xanthan gum, tara gum, carrageenan;
silicate derivatives such as magnesium aluminium silicates;
cellulose derivatives such as hydroxypropyl cellulose, microcrystalline cellulose;
polyacrylic acid based derivatives such as sodium polyacrylate, Acrylates/C10-30 Alkyl Acrylate Crosspolymer, carbomer, Acrylates/Beheneth-25 Methacrylate Copolymer, Hydroxyethyl Acrylate/Sodium Acryloyldimethyl Taurate Copolymer; and
starch derivatives such as hydroxypropyl starch phosphate.

In an embodiment, exemplary thickeners used to obtain an oil gel or to increase the film formation of a lipophilic or lipo-alcoholic monophase may include one or more of:
silica derivatives such as silica, dimethyl silica silylate; and
Poly C10-30 Alkyl Acrylate.

In an embodiment, a oil-in-water fluid (or lotion formulation type) may include a formulation with a viscosity of up to 20,000 mPa·s, preferably up to 15,000 mPa·s measured at 25° C. with a Brookfield DVIII type device at a rotation per minute of 10 (10 rpm) using a spindle RV5. When the formulation is too fluid to be measured with a RV5 spindle, a spindle LV3 might be necessary to be used.

In an embodiment, a cream may include a formulation which is visually (by naked eyes) firm, which does not flow from a bottle with a viscosity above 20,000 mPa·s measured at 25° C. for example with a Brookfield DVIII type device at a rotation per minute of 10 (10 rpm) using a spindle RV5 or RV6.

In an embodiment, the input unit may include without limitation any item or element forming a boundary configured for transferring information. In particular, the input unit may be configured for transferring information onto a computational device, e.g. onto a computer, such as to receive information. The input unit preferably is a separate unit configured for receiving or transferring information onto a computational device, e.g. one or more of: an interface, specifically a web interface and/or a data interface; a keyboard; a terminal; a touchscreen, or any other input device deemed appropriate by the skilled person. More preferably, the input unit comprises or is a data interface configured for transferring or exchanging information as specified herein below.

In an embodiment, the output unit may include without limitation any item or element forming a boundary configured for transferring information. In particular, the output unit may be configured for transferring information from a computational device, e.g. a computer, such as to send or output information, e.g. onto another device, e.g. a control unit, that controls and/or monitor the production process of the produced composition. The output unit preferably is a separate unit configured for outputting or transferring information from a computational device, e.g. one or more of: an interface, specifically a web interface and/or a data interface; a screen, a printer, or a touchscreen, or any other output device deemed appropriate by the skilled person. More preferably, the output unit comprises or is a data interface configured for transferring or exchanging information as specified herein below.

Preferably, the input unit and the output unit are configured as at least one or at least two separate data interface(s); i.e. preferably, provide a data transfer connection, e.g. a wireless transfer, an internet transfer, Bluetooth, NFC, inductive coupling or the like. As an example, the data transfer connection may be or may comprise at least one port comprising one or more of a network or internet port, a USB-port and a disk drive. The input unit and/or the output unit may also be may be at least one web interface.

In an embodiment, the processing unit may include, without limitation, to an arbitrary logic circuitry configured for performing operations of a computer or system, and/or, generally, to a device or unit thereof which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. The processing unit may be configured for pre-processing the input data. The pre-processing may comprise at least one filtering process for input data fulfilling at least one quality criterion. For example, the input data may be filtered to remove missing variables.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 illustrates a block diagram of an exemplary apparatus for determining a performance of a sunscreen product comprising at least one UV filter.

FIG. 2 schematically depicts an exemplary computer network environment or implementing embodiments of the present disclosure.

FIG. 3 illustrates a flowchart describing a computer-implemented method for determining a performance of a sunscreen product comprising at least one UV filter.

FIG. 4A illustrates an exemplary implementation of the interactive user interface that enable an evaluation of the interaction between the formulation types and the performance index.

FIG. 4B illustrates another exemplary implementation of the interactive user interface that enable an evaluation of the interaction between the formulation types and the performance index.

FIG. 4C illustrates a further exemplary implementation of the interactive user interface that enable an evaluation of the interaction between the formulation types and the performance index.

FIG. 5A illustrates curves of the shape parameter c for different formulation types, in which the amplitude of the function is not unambiguously related to a performance of a formulation type in terms of the performance index of the sunscreen product.

FIG. 5B illustrates curves of the shape parameter c for different formulation types, in which the amplitude of the function is related to a performance of a formulation type in terms of the performance index of the sunscreen product.

FIG. 6 illustrates an example of the curves of the shape parameter c plotted against the TFC given by equations (6) and (8) described below.

FIG. 7 illustrates a flowchart describing a method for providing manufacturing a sunscreen product.

FIG. 8 shows an example of a flowchart describing a method for validating manufacture of a sunscreen product.

FIG. 9 shows an example of a production line for manufacturing a sunscreen product with a monitoring apparatus.

FIG. 10 shows another example of a production line for manufacturing sunscreen product with a validation apparatus

### DETAILED DESCRIPTION OF EMBODIMENTS

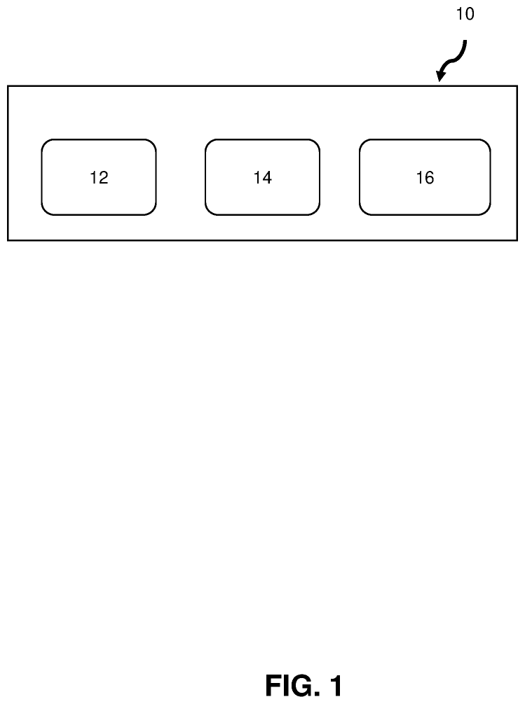

FIG. 1 illustrates a block diagram of an exemplary apparatus 10 for determining a performance of a sunscreen product comprising at least one UV filter. The apparatus 10 includes an input unit 12, a processing unit 14, and an output unit 16.

In general, the apparatus 10 may comprise various physical and/or logical components for communicating and manipulating information, which may be implemented as hardware components (e.g. computing devices, processors, logic devices), executable computer program instructions (e.g. firmware, software) to be executed by various hardware components, or any combination thereof, as desired for a given set of design parameters or performance constraints. Although FIG. 1 may show a limited number of components by way of example, it can be appreciated that a greater or a fewer number of components may be employed for a given implementation.

In some implementations, the apparatus 10 may be embodied as, or in, a device or apparatus, such as a server, workstation, or mobile device. The apparatus 10 may comprise one or more microprocessors or computer processors, which execute appropriate software. The processing unit 14 of the apparatus 10 may be embodied by one or more of these processors. The software may have been downloaded and/or stored in a corresponding memory, e.g. a volatile memory such as RAM or a non-volatile memory such as flash. The software may comprise instructions configuring the one or more processors to perform the functions described herein.

It is noted that the apparatus 10 may be implemented with or without employing a processor, and also may be implemented as a combination of dedicated hardware to perform some functions and a processor (e.g. one or more programmed microprocessors and associated circuitry) to perform other functions. For example, the functional units of the apparatus 10, e.g. the input unit 12, the one or more processing units 14, and the output unit 16 may be implemented in the device or apparatus in the form of programmable logic, e.g. as a Field-Programmable Gate Array (FPGA). In general, each functional unit of the apparatus may be implemented in the form of a circuit.

In some implementations, the apparatus 10 may also be implemented in a distributed manner. For example, some or all units of the apparatus 10 may be arranged as separate modules in a distributed architecture and connected in a suitable communication network, such as a 3rd Generation Partnership Project (3GPP) network, a Long Term Evolution (LTE) network, Internet, LAN (Local Area Network), Wireless LAN (Local Area Network), WAN (Wide Area Network), and the like.

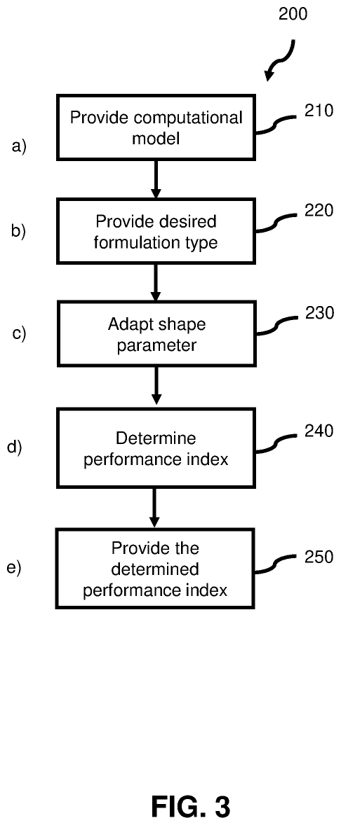

The processing unit(s) 14 may execute instructions to perform the method described herein, which will be explained in detail with respect to the example shown in FIG. 3.

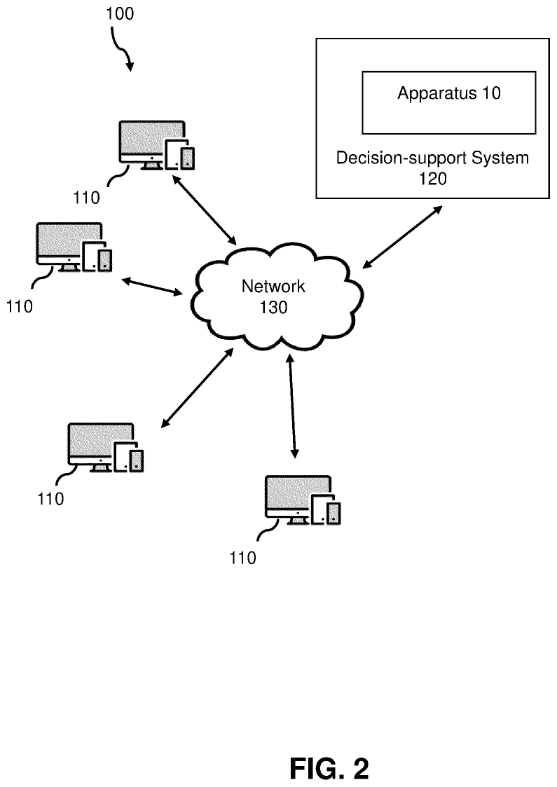

FIG. 2 schematically depicts an exemplary computer network environment 100 or implementing embodiments of the present disclosure. As illustrated, the system 100 includes a plurality of electronic communication devices 110, a decision-support system 120, and a network 130. While four electronic communication devices 110 are illustrated, any number of electronic communication devices 110 may be in use.

A query may be transmitted from the electronic communication devices 110 through the network 130 to the decision-support system 120 to perform a calculation of one or more sun protection indices of a UV filter composition. The query may include the information about a desired formulation type of the UV filter composition. In the example of FIG. 2, the apparatus shown in FIG. 1 may be embodied as, or in, the decision-support system 120. The apparatus 10 processes the query and determines performance indices of the sunscreen product. Results of the query are returned from the decision-support system 120 through the network 130 to the electronic communication device 110. While a single decision-support system 120 is illustrated in FIG. 2 by way of example, it should be appreciated that the functionality of the decision-support system 120 may be distributed over multiple servers, which may be clustered, geographically distributed across the network 130, or any combination thereof.

The electronic communication devices 110 may act as terminals, graphical display clients, or other networked clients to the decision-support system 120. The electronic communication devices 110 may comprises an application configured to interface with the web service provided by the decision-support system 120. For example, a web browser application at the electronic communication devices 110 may support interfacing with a web server application at the decision-support system 120. Such a browser may use controls, plug-ins, or applets to support interfacing to the decision-support system 120. The electronic communication devices 110 may use other customized programs, applications, or modules to interface with the decision-support system 120. The electronic communication devices 110 may be desktop computers, laptops, handhelds, mobile devices, mobile telephones, servers, terminals, thin-clients, or any other computerized devices.

The network 130 may be any communications network capable of supporting communications between the electronic communication device 110 and the decision-support system 120. The network 130 may be wired, wireless, optical, radio, packet switched, circuit switched, or any combination thereof. The network 130 may use any topology, and links of the network 130 may support any networking technology, protocol, or bandwidth such as Ethernet, DSL, cable modem, ATM, SONET, MPLS, PSTN, POTS modem, PONS, HFC, satellite, ISDN, WiFi, WiMax, mobile cellular, any combination thereof, or any other data interconnection or networking mechanism. The network 130 may be an intranet, the Internet (or the World Wide Web), a LAN, WAN, MAN, or any other network for interconnecting computers. To support high volume and load, a distributed computing environment may be implemented by using networking technologies that may include, but are not limited to, TCP/IP, RPC, RMI, HHTP, Web Services (XML-RPC, JAX-RPC, SOAP, etc.).

It should be appreciated that, in addition to the illustrated network environment shown in FIG. 2, the decision-support system 120 and the electronic communication device 110 may be combined into a single computing device. Such a combined computing device can support a calculation of various sun protection indices of a UV filter composition.

FIG. 3 illustrates a flowchart describing a computer-implemented method 200 for determining a performance of a sunscreen product comprising at least one UV filter.

At block 210, i.e., step a), a computational model is provided to calculate a performance index of a sunscreen product. The computational model uses the properties of the UV filter(s) (e.g., extinction, photostability alone, or photostability of a combination thereof) and applies a gamma distribution as a model to determine a film thickness distribution of the sunscreen product. In some examples, the computational model may be stored in the decision-support system 120 illustrated in FIG. 2. The electronic communication devices 110 may comprise a web browser application or other customized programs, applications, or modules configured to interface with the web service provided by the decision-support system 120. Via the web browser application or other customized programs, applications, or modules, the user may access or retrieve the computational model in the decision-support system 120 using e.g., Username and Password Authentication.

The performance index may include SPF and/or UVA-PF. For example, the basic principle of SPF calculations is the calculation of the factor by which the intensity of the UV radiation is reduced due to the presence of a sunscreen. This factor is given by the inverse of the UV transmittance of the absorbing film, 1/T. At a certain wavelength λ, (1/T(λ) is also designated as monochromatic protection factor (MPF). As the spectral range relevant for the formation of erythema is between 290 nm and 400 nm, the monochromatic protection factors should be averaged over this range. In order to obtain the SPF, this average should be weighted with the intensity of the light source, Sₛ(λ), and the erythemal action spectrum, Sₑᵣ(λ), leading to the following equation:

$$SPF = \frac{{\sum }_{290}^{400} {S}_{e r} ( λ ) {S}_{s} ( λ )}{{\sum }_{290}^{400} {S}_{e r} ( λ ) {S}_{s} ( λ ) T ( λ )} \qquad ( 1 )$$

Data for Sₛ(λ) and Sₑᵣ(λ) are available e.g., in D. L. Diffey, J. Robson, J. Soc. Cosmet, Chem. 40, 127 (1989) and A. F. Mckinlay, B. L. Diffey, CIE J. 6 17 (1987). However, T(λ) has to be determined for the respective sunscreen either by in vitro measurement or by calculation.

For example, the basic principle of UVA-PF calculations (or PPD calculation) is similar to the calculation of the SPF and differs with the used spectral range and used action spectrum. The spectral range relevant for the protection against UVA rays is between 320 nm and 400 nm. The monochromatic protection factors should be averaged over this range. In order to obtain the UVA-PF, this average should be weighted with the intensity of the light source, Sₛ(λ), and the persistence pigment darkening action spectrum, S_{PPD}(λ), leading to the following equation:

$$UVA - PF ( PPD ) = \frac{{\sum }_{320}^{400} {S}_{P P D} ( λ ) {S}_{s} ( λ )}{{\sum }_{320}^{400} {S}_{P P D} ( λ ) {S}_{s} ( λ ) T ( λ )} \qquad ( 2 )$$

When calculating the UV transmittance T(λ), an irregular film thickness distribution must be considered, since it is not possible to apply a sunscreen product uniformly e.g., with the same thickness, over the whole human skin since human skin shows itself a certain roughness. This is of prime importance since the optical transmittance of an absorbing film of uniform thickness is lower compared to that of a corresponding irregular film of the same average thickness.

Several models have been published to describe sunscreen film irregularity, such as the model of Ferrero and coworkers, which is published in e.g., L. Ferrero, M. Pissavini, S. Marguerie, L. Zastrow. J. Cosmet, Sci, 54, 63 (2003). In this model, the film profile becomes equivalent to the bearing area curve of Abbot and Firestone and is constructed based on a cumulative distribution function F(h) containing the film height has a random variable. A gamma law may represent an asymmetrical distribution, and ƒ(h) is the associated probability density function:

$$f ( h ) = {( \frac{h}{b} )}^{c - 1} {e}^{- ( \frac{h}{b} )} \frac{1}{b Γ ( c )} \qquad ( 3 )$$

where his the random variable “relative height”, c is the shape parameter to be adjusted, bis needed for normalization, and Γ(c) is the value of the gamma function at c. The cumulative height distribution F(h) is obtained by integration of ƒ(h):

$$F ( h ) = {\int }_{0}^{∞} f ( h ) d h \qquad ( 4 )$$

In order to construct the film thickness profile, his deduced from its cumulative distribution F(h), ranging from 0 to 1. The transmittance of the film can then be calculated:

$${T}_{film} ( λ ) = {\int }_{0}^{1} 1 {0}^{- \overline{ε} ( λ ) \overline{c} d h ( F )} d F \qquad ( 5 )$$

The transmittance calculated via equation (5) can be fed into equation (1) or equation (2), resulting a calculated SPF value (referred to as SPF in silico) or a calculated UVA-PF value (referred to as UVA-PF in silico). The shape parameter cis the screw by which the calculated result can be adjusted to fit the SPF in vivo and/or SPF in vitro.

At block 220, i.e., step b), a desired formulation type of the sunscreen product is provided. A graphical user interface may be used to provide an interactive user interface allowing the user to select one or more desired formulation types.

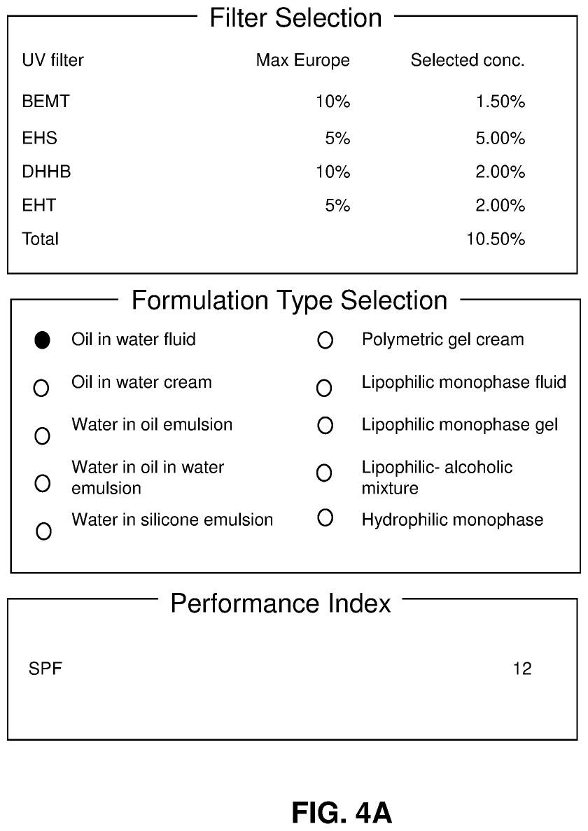

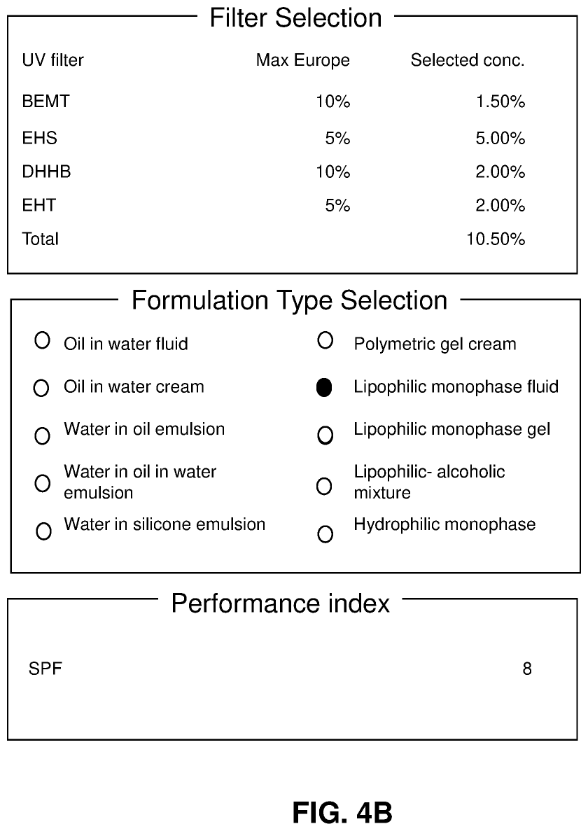

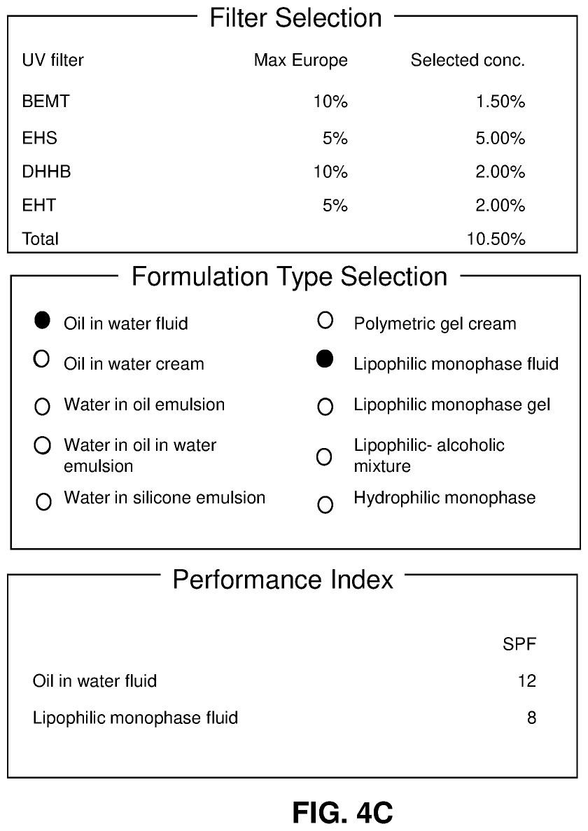

FIGS. 4A to 4C illustrate an exemplary implementation of the interactive user interface that enable an evaluation of the interaction between the formulation types and the performance index. The interface of the illustrated example displays a plurality of facets. For example, the facets depicted are “Filter Selection”, “Formulation Type Selection”, and “Performance Index”. In the examples shown in FIGS. 4A to 4C, the performance index is SPF. The layout, number, and order of the facets and the specific names of the facets are presented solely to illustrate the concept. Other layouts, numbers, orders, or names of facets may of course by dynamically displayed.

It should be appreciated that although not shown in FIGS. 4A to 4C, the user may have the opportunity to select and define cosmetic ingredients of the sunscreen formulation, such as emulsifiers, emulsifying polymers, emollients, hydrophilic humectants, waxes, thickening polymers, consistency enhancers, silicon based compounds, pH regulators, actives, preservatives, and perfumes. One or more facets (not shown) may be provided to allow the user to select one or more desired ingredients and to define the percentage of the selected ingredient(s).

Under the facet “Filter Selection”, the user has the opportunity to select one or more UV filters and define the percentage of each UV filter. In the examples shown in FIGS. 4A to 4C, the following UV filters are selected: BEMT (INCI: Bis-ethylhexyloxyphenol methoxyphenyl triazine), DHHB (INCI: Diethylamino hydroxybenzoyl hexyl benzoate), EHS (INCI: Ethylhexyl Salicylate), and EHT (INCI: Ethylhexyl Triazone).

Under the facet “Formulation Type Selection”, the user has the opportunity to select one or more formulation types. Examples of the formulation types may include, but are not limited to, oil-in-water fluid emulsion, oil-in-water cream emulsion, water-in-oil emulsion, oil-in-water-in-oil emulsion, water-in-oil-in-water emulsion, water-in-silicone emulsion, silicone-in-water emulsion, polymeric gel cream, lipophilic monophase oil, lipophilic monophase gel, lipophilic monophase stick, lipophilic-alcoholic mixture, hydrophilic monophase fluid, hydrophilic monophase gel, and powder.

As depicted in the example presented in FIGS. 4A to 4C, the value of the SPF is presented in the interface in response to the user-defined UV-filter composition and the selected formulation type.

Turning back to FIG. 3, at block 230, i.e., step c), the shape parameter c of the gamma distribution in equation (3) is adapted to the desired formulation type. The shape parameter c in equation (3) is not a simple constant, but a function of the TFC. The function is a saturation like function with a larger slope at a lower TFC and a smaller slope at a higher TFC, and the amplitude of the function is related to a performance of a formulation type in terms of the performance index of the sunscreen product.

In an example, the saturation like function may be given by equation (6):

$$c = a 1 + a 2 \cdot ( \frac{{TFC}^{a 3}}{a 4 + {TFC}^{a 3}} ) \qquad ( 6 )$$

with a1, a2, a3, a4 and a5 as adjustable parameters. These parameters are determined for each formulation type.

In another example, the saturation like function may be given by equation (7):

$$c = a 1 + a 2 \cdot ( \frac{{TFC}^{a 3}}{a 4 + {TFC}^{a 3}} ) + \frac{a 5}{4} - a 5 \cdot {( REAE - 0.5 )}^{2} \qquad ( 7 )$$

with a1, a2, a3, a4 and a5 as adjustable parameters. Compared to equation (6), equation (7) further comprises a parameter a5, which is only needed when the filter composition contains at the same time UV absorbers in the oil and in the water phase, where REAE means the relative erythemally active extinction in the oil-phase and relates to the increase of the performance index that can be obtained when filters are present at the same time in the oil and in the water phase of an emulsion. These parameters are determined for each formulation type. If the filter systems of the formulations do contain only filters in the water phase or only filters in the oil phase, the parameter a5 may be neglected.

Preferably, the adjustable parameter a1 for the shape parameter c is in the range of from 0.35 to 0.60, the adjustable parameter a2 for the shape parameter c is in the range of from 0.30 to 0.68, the adjustable parameter a3 for the shape parameter c is in the range of from 0.90 to 1.50, the adjustable parameter a4 for the shape parameter cis below 8, preferably below 6 and most preferably below 4, the adjustable parameter a5 for the shape parameter cis in the range of from 0 to 1.

A further example of the saturation like function may be given by equation (8):

$$c = a 1 + a 2 \cdot ( 1 - {e}^{( - TFC \cdot a 6 )} ) \qquad ( 8 )$$

with a1, a2, a6 as adjustable parameters, which are determined for each formulation type. In equation (8) the parameters a1 and a2 have the same meaning as described with respect to equations (6) and (7). Parameter a6 is decisive for the steepness of the function. The parameters a1 and a2 are in the same ranges as described with respect to equations (6) and (7), and a6 is between 0.1 and 0.9.

A still further example of the saturation like function may be given by equation (9):

$$c = a 1 + a 2 \cdot ( 1 - {e}^{( - TFC \cdot a 6 )} ) + \frac{a 5}{4} - a 5 \cdot {( REAE - 0.5 )}^{2} \qquad ( 9 )$$

with a1, a2, and a6 as adjustable parameters, which are determined for each formulation type. In equation (9) the parameters a1, a2, and a6 have the same meaning as described with respect to equation (8), and the parameter a5 has the same meaning as described with respect to equation (7). The parameters a1, a2, and a6 are in the same ranges as described with respect to equation (8), and 0≤a5≤1.

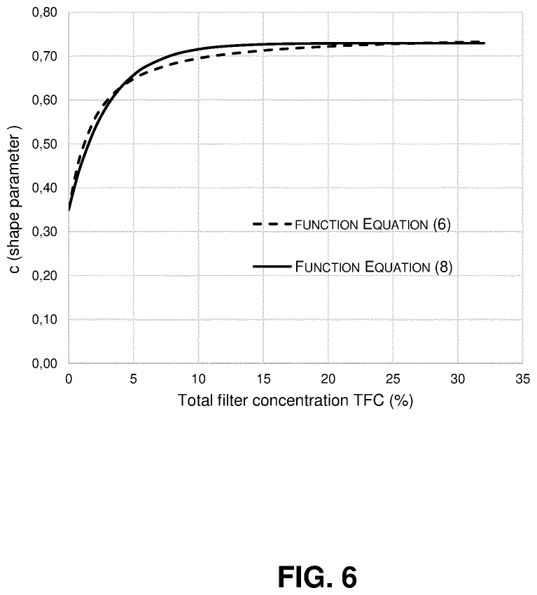

FIG. 6 shows an example of the curves of the shape parameter c plotted against the TFC given by equations (6) and (8). As shown in FIG. 6, the curves of the shape parameter c plotted against the TFC for both equations have a larger slope at a lower TFC and a smaller slope at a higher TFC. The determined parameters for the saturation like function (6) are a1=0.40, a2=0.62, a3=1.09, and a4=1.95. The determined parameters for the saturation like function (8) are a1=0.37, a2=0.62, and a6=0.33.

The above exemplary saturation like functions describing the shape parameter c as a function of the TFC may be predetermined for each of the different formulation types using appropriate formulations with a range of known SPF in vivo values used as references.

As an example, in equation (7), different formulation types having a range of known SPF in vivo values are provided as references. The four parameters a1 to a4 were determined for each formulation type to get the best correlation between the SPF in silico based on the determined a1 to a4 parameters with the SPF in-vivo of these reference formulations. The parameter a5 was set to a value of 0.5 in this case. When the filter system contains only filters in the water phase or inversely only filters in the oil phase the second term in Equation (7) containing the parameter a5 will be zero. For this purpose, filter combinations with expected SPF values of SPF 50, SPF 30 and SPF 15 (calculated with the sunscreen simulator of BASF (https://sunscreensimulator.basf.com/Sunscreen_Simulator/login) were incorporated in different formulation types (or formulation vehicles) and the performance of each of these formulation type containing each filter combination (SPF expected 50, SPF expected 30 and SPF expected 15) was measured in vivo in accordance with ISO24444 in two different test institutes and twice in each institute. As an example, the obtained SPF in vivo of a water-in-oil formulation type with an expected SPF 15 is the average of four individual SPF measurements according to ISO24444. The SPF in vivo data obtained for all these reference formulation types (or formulation vehicles) was used to determine the shape parameter c based on the determination of the values of parameters a1 to a4 for each formulation type, that can then be used to predict the performance of any other filter combination intended to be incorporated in one of the formulation type (or formulation vehicle).

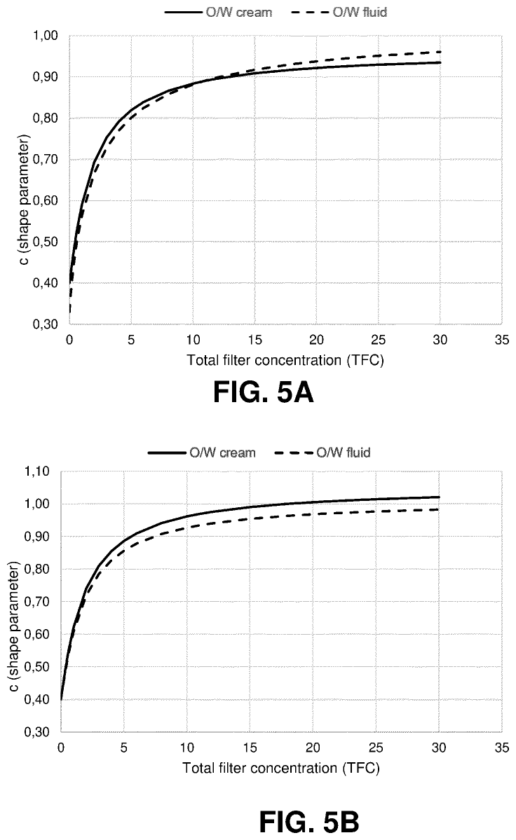

However, as shown in FIG. 5A, when plotting the shape parameters c for different formulation types against the Total filter concentration (TFC), an unexpected issue was discovered. The curves of the shape parameter c plotted against the TFC for different formulation types intersect with one another, such as oil-in-water cream (O/W cream) and oil-in-water fluid (O/W fluid) shown in FIG. 5A. However, since the SPF of a certain formulation type should always be higher than that for another formulation type over the whole TFC range, the curves of the shape parameter c as function of the TFC should not intersect for different formulation types.

Surprisingly, it was possible to overcome this problem by applying the following strategy. The values for a1, a3, a4, and a6 were kept at a fixed value irrespective of the formulation type (or formulation vehicle) and only the value for a2 differed for each formulation type. In this manner, it was ensured that the curves for the shape parameter c of different formulation types plotted against the TFC did not intersect with one another, such as O/W cream and O/W fluid shown in FIG. 5B. With this approach, it was possible to determine the value of the shape parameter c for each formulation type (or formulation vehicle) which provided compared to previously known in silico methods an improved accuracy for the resulting SPF in silico which closely matched the SPF in vivo for a set of reference formulations

Therefore, the adjustable parameters a1, a3, a4 and a6 have the same value for each formulation type (or formulation vehicle) while the adjustable parameter a2 differs depending on the formulation type. In other words, the amplitude of the saturation like function is related to a performance of a formulation type in terms of the performance index such as the SPF of the sunscreen product. In this manner, the problem of an intersection of the curve of the shape parameter c when plotted against the TFC was overcome.

The different parameters a1 to a4 influence the curve of the shape parameter c in different ways. The parameter a1 determines the minimum value of the shape parameter c. Preferably, it does not undercut a certain value because it can impact the simulation for filter combinations with low TFC and could lead to an underestimation of the SPF value for those combinations. For this reason, it is preferred that the value of the parameter a1 is in the range of from 0.35 to 0.60.

The parameters a3, a4 and a6 determine the form of the function of the shape parameter c plotted against the TFC that means they impact the steepness of c as function of the TFC. If the steepness is too high, the SPF simulated may be overestimated at low TFC and underestimated at high TFC. The two parameters a3 and a4 impact the steepness inversely. To get the most realistic SPF predictions from low to very high SPF values, the value of the parameter a3 should preferably be a value from 0.90 to 1.50 and a4 should preferably be below 8, more preferably below 6, and even more preferably below 4, the value of the parameter a6 should preferably be a value from 0.1 to 0.9.

The parameter a2 determines the amplitude of the curve of the shape parameter and therefore the range of the calculated SPF (SPF in silico) values. When a2 is too low, it is possible that all calculated SPF irrespective of the TFC could be too small and vice versa. To get the most realistic and accurate SPF predictions from low to very high SPF values, the value of the parameter a2 should preferably be in the range of from 0.30 and 0.68.

For example, the adjustable parameters a1 to a5 of the saturation like function for the shape parameter c (equation (7)) were predetermined for the reference formulation types “oil-in-water lotion” and “oil-in-water cream” using known values of SPF in vivo for different expected SPFs. (SPF 50, SPF 30, SPF 15) The determined parameters for the saturation like function were a1=0.40, a2=0.62, a3=1.09, and a4=1.95 and a5=0.5 for the oil-in-water lotion resulting in a value of c=1.0376, and a1=0.40, a2=0.64, a3=1.09, and a4=1.95 and a5=0.5 for the oil-in-water cream emulsion resulting in a shape parameter c of 1.0542.

The resulting shape parameters c (i.e., c values) are then used for prediction of the SPF in silico of any UV filter combinations for each of the different formulation types (or formulation vehicles).

Table 1 compares the SPF in silico using values of a1 to a4 outside the preferred ranges (shown as COMP 1 in Table 1) and the SPF in silico using values of a1 to a4 within the preferred ranges (shown as Cream INV1 in Table 1) for different compositions of cream formulations. In particular, in the COMP 1 simulation, a1=0.33 that is outside of the range of from 0.35 to 0.60, a2=0.69 that is outside of the range of from 0.30 and 0.68, a3=0.89 that is outside of the range of from 0.90 to 1.50, a4=1.95, and a5=0.50. Contrary to COMP 1 simulation, in the Cream INV1 simulation for different compositions of cream formulations, a1=0.40, a2=0.64, a3=1.09, a4=1.95, a5=0.50. These parameters all fall into the above-described preferred ranges. As can be seen from Table 1, compared to the SPF in silico obtained with parameters of COMP 1, the SPF in silico obtained with parameters of INV1 is closer to the reference SPF in vivo and within +/−15% of SPF in vivo for different compositions of cream formulations. The prediction of SPF is more accurate for creams with INV1.

TABLE 1

| | | Cream | Cream | Cream | Cream | Cream | Cream | Cream | Cream |
|---|---|---|---|---|---|---|---|---|---|
| | P6 | Form | Form | Form | Form | Form | Form | Form | Form |
| | cream⁽³⁾ | 2 | 3 | 4 | 5 | 6 | 7 | 8 | 9 |
| BEMT | 2 | 1.5 | 3.5 | | 1 | 2.2 | 1.5 | 2 | |
| MBBT | 6 | 2.5 | | 3 | | | | 3 | |
| EHMC | 5 | | | 7.5 | | | | | |
| BMDBM | | 4 | | | | 5 | | 3 | |
| OCR | | 10 | | | | | | | |
| EHS | | 5 | 5 | 5 | 5 | 3.2 | 5 | | 4.9 |
| DHHB | | | 10 | 8 | 6.5 | | 2 | | 5 |
| BEMT aq | | | 2 | | | | | | |
| EHT | | | 3.5 | 3 | 3 | 3.2 | 2 | 3.5 | 3.5 |
| TBPT | | | | | 2.5 | | | | |
| PBSA | | | | | 1.5 | 1.2 | | | |
| DBT | | | | | | 1.8 | | | |
| TiO2* | | | | | | | | | 4 |
| SPF in | 43 ⁽¹⁾ | 41 ⁽²⁾ | 66⁽²⁾ | 62⁽²⁾ | 61⁽²⁾ | 48⁽²⁾ | 22⁽²⁾ | 35⁽²⁾ | 39⁽²⁾ |
| vivo | | | | | | | | | |
| SPF in | 36.5-49.5 | 35.9-47.2 | 56.1-75.9 | 52.7-71.3 | 51.9-70.2 | 40.8-55.2 | 18.7-25.3 | 29.8-40.3 | 33.2-44.9 |
| vivo +/− | | | | | | | | | |
| 15% | | | | | | | | | |
| SPF in | 34 | 35 | 49 | 51 | 54 | 34 | 17 | 30 | 35 |
| silico | | | | | | | | | |
| using | | | | | | | | | |
| COMP 1 | | | | | | | | | |
| a1 = 0.33 | | | | | | | | | |
| a2 = 0.69 | | | | | | | | | |
| a3 = 0.89 | | | | | | | | | |
| a4 = 1.95 | | | | | | | | | |
| a5 = 0.50 | | | | | | | | | |
| SPF in | 39 | 40 | 57 | 58 | 62 | 39 | 20 | 35 | 41 |
| silico | | | | | | | | | |
| using | | | | | | | | | |
| Cream | | | | | | | | | |
| INV1 | | | | | | | | | |
| a1 = 0.40 | | | | | | | | | |
| a2 = 0.64 | | | | | | | | | |
| a3 = 1.09 | | | | | | | | | |
| a4 = 1.95 | | | | | | | | | |
| a5 = 0.5 | | | | | | | | | |

Table 2 compares the SPF in silico using values of a1 to a4 outside the preferred ranges as the comparative set (shown as COMP 1 in Table 2) and the SPF in silico using values of a1 to a4 within the preferred ranges (shown as Lotion INV2 in Table 2) for different compositions of lotion formulations (oil in water fluid emulsions). In Table 2, the COMP 1 simulation uses the same values of a1 to a4 of Table 1. In the Lotion INV2 simulation, since the adjustable parameters a1, a3, and a4 have the same value for each formulation type and only the adjustable parameter a2 differs depending on the formulation type, in the Lotion INV 2 simulation (in table 2) the values of parameters a1, a3, and a4 are the same as for the Cream INV1 simulations shown in Table 1 while the adjustable parameter a2 differs, e.g., a2=0.64 for Cream INV1 simulation shown in Table 1 and a2=0.62 for Lotion INV 2 simulation shown in Table 2. As can be seen from Table 2, compared to the SPF in silico obtained with parameters of COMP 1, the SPF in silico obtained with parameters of Lotion INV2 simulation is also closer to SPF in vivo and within +/−15% of SPF in vivo for different compositions of lotion formulations. The prediction of SPF is more accurate for lotions with INV2.

TABLE 2

| | P5 | Lotion | Lotion | Lotion | Lotion | Lotion | Lotion |
|---|---|---|---|---|---|---|---|
| | lotion ⁽³⁾ | Form 1 | Form 2 | Form 3 | Form 4 | Form 5 | Form 6 |
| BEMT | | 2 | 2 | | 2.5 | | 3 |
| MBBT | | 4 | | | | 3.5 | |
| EHMC | | | | 10 | | | |
| BMDBM | | 2 | | | | | |
| OCR | | | | | | | |
| EHS | | | | | | | 5 |
| DHHB | | | 8 | 8 | 6.5 | 3 | 6 |
| BEMT aq | | | 2 | 2 | | | 2.5 |
| EHT | | 2 | 3 | 1.5 | 2.5 | 1 | 3 |
| TBPT | | | | | 4 | 0.25 | |
| PBSA | | | 3 | | | | |
| DBT | | | | | | | 3 |
| ZnO* | | | | 5 | | | |
| SPF in vivo | 30.6 ⁽¹⁾ | 31⁽²⁾ | 60⁽²⁾ | 53⁽²⁾ | 66⁽²⁾ | 20⁽²⁾ | 62⁽²⁾ |
| SPF in | 26-35.2 | 26.4-35.7 | 51-69 | 45.1-61 | 56.1-75.9 | 17-23 | 52.7-71.3 |
| vivo +/− 15% | | | | | | | |
| SPF in silico | 28 | 26 | 52 | 48 | 53 | 16 | 57 |
| using COMP | | | | | | | |
| 1 | | | | | | | |
| a1 = 0.33 | | | | | | | |
| a2 = 0.69 | | | | | | | |
| a3 = 0.89 | | | | | | | |
| a4 = 1.95 | | | | | | | |
| a5 = 0.50 | | | | | | | |
| SPF in silico | 30 | 29 | 58 | 53 | 60 | 18 | 63 |
| using Lotion | | | | | | | |
| INV2 | | | | | | | |
| a1 = 0.40 | | | | | | | |
| a2 = 0.62 | | | | | | | |
| a3 = 1.09 | | | | | | | |
| a4 = 1.95 | | | | | | | |
| a5 = 0.50 | | | | | | | |

ZnO, Zinc Oxide
*refers to the active amount of the market product Z-Cote HP1. Since each Zinc Oxide grade (different market products) might have a slight different absorption spectrum due for example to the different coatings, the absorption spectrum of each Zinc oxide grade can be considered in the computational model to get a better accuracy of the calculated performance.
⁽¹⁾ Annex c in ISO24444
⁽²⁾ISO 24444 test performed in 1 external institute, screening test
⁽³⁾ reference sunscreen formulation in annex c in ISO24444

We tested the correlation, given as an equation in table 3, between SPF in silico and SPF in vivo of a series of different formulation types using the values of the adjustable parameters in the above-described preferred ranges and the values of the adjustable parameters of the comparative set (COMP 1 in table 1 and table2).

In Table 3 the slope of the equation testing the relationship between SPF in silico and SPF in vivo is close to 1 using the adjustable parameters within the above-described preferred ranges compared to the values of the adjustable parameters of the comparative test (COMP 1), thereby showing a better correlation and prediction of the performance with the above-described preferred ranges. In Table 3, y corresponds to the SPF in silico, and x corresponds to the SPF in vivo.

TABLE 3

| | COMP 1 | INV |
|---|---|---|
| | a1 = 0.33 | a1: 0.35 to 0.6 |
| | a2 = 0.69 | a2: 0.3 to 0.68 |
| | a3 = 0.89 | a3: 0.9 to 1.5 |
| | a4 = 1.95 | a4: <4 |
| | a5 = 0.50 | a5: 0 to 1 |
| Sunscreen cream | y = 0.9048x | y = 1.0024x |
| formulations (65 different | | |
| cream formulations in total) | | |
| Sunscreen oil in water fluid | y = 0.9625x | y = 0.9918x |
| formulations (29 different | | |
| lotion formulations in total) | | |
| Sunscreen oils (6 different | y = 1.9519x | y = 1.0586x |
| oil formulations in total) | | |

Accordingly, the shape parameter c in equation (3), which describes the thickness distribution layer of an applied sunscreen, is adaptable according to the user-defined formulation type (or formulation vehicle), so that the determined SPF in silico considers not only the properties of the UV filters and their interactions but also the formulation type (e.g., oil-in-water fluid, oil-in-water cream, etc.) of the sunscreen product the UV filter combination is intended to be used in. For different formulation types (or formulation vehicles), the adjustable parameters (a1 to a6) in the function for the shape parameter c as given in equation (6), (7) or (8) may be determined based on in vitro and/or clinical in vivo experimental data. For example, SPF values in vivo for different reference formulation types including the same UV filter combination can be measured, and the shape parameter c can be determined using these experimental in vivo data such that the SPF in silico is equal or as close as possible to SPF in vivo. The experiment may be done over a range of different SPF values e.g., to cover the low (e.g., SPF≤10) to very high SPFs (e.g., SPF=50+) and to increase the accuracy of the prediction. In other words, several UV-filter combinations aiming different SPF values have been tested in the different formulation types to obtain the shape parameters c for each formulation type.

Turning back to FIG. 3, at block 240 in FIG. 3, i.e., step d), the performance index (e.g., SPF) of a UV-filter composition is determined for the desired formulation type utilizing the adapted shape parameter c of the Gamma distribution. The value of the SPF may be determined using equation (1). The shape parameter c in equation (3) is adaptable in response to a change of the user-selected formulation type.

For example, as shown by way of example in FIG. 4A, the user may select “oil in water fluid emulsion” as the desired formulation type. In response to the user-defined or user-selected formulation type, the SPF value of 12 is calculated using the shape parameter c defined for the oil in water fluid formulation type and presented under the facet “Performance Index”. In the example depicted in FIG. 4B, the user may select “lipophilic monophase fluid” as the desired formulation type. In response to the user-defined or user-selected formulation type, the SPF value of 8 is determined using the shape parameter c defined for a “lipophilic monophase fluid” formulation type and presented under the facet “Performance Index”. As the effect of formulation type is considered, different formulation types shown in FIGS. 4A and 4B have different SPF in silico values even with the same UV filter composition. In some examples, the user may have the opportunity to select two or more formulation types. As shown by way of example in FIG. 4C, the user may select multiple formulation types, such as “oil in water fluid” and “lipophilic monophase fluid”. The SPF values for these formulation types are then calculated and presented under the facet “Performance Index”.

Turning back to FIG. 3, at block 250, i.e., step e), the determined performance index of the sunscreen product having the desired formulation type is provided, which is preferably usable for manufacturing the sunscreen product. For example, the determined performance index may be returned from the decision-support system 120 through the network 130 to the electronic communication device 110. For example, the determined performance index may be stored in the decision-support system 120.

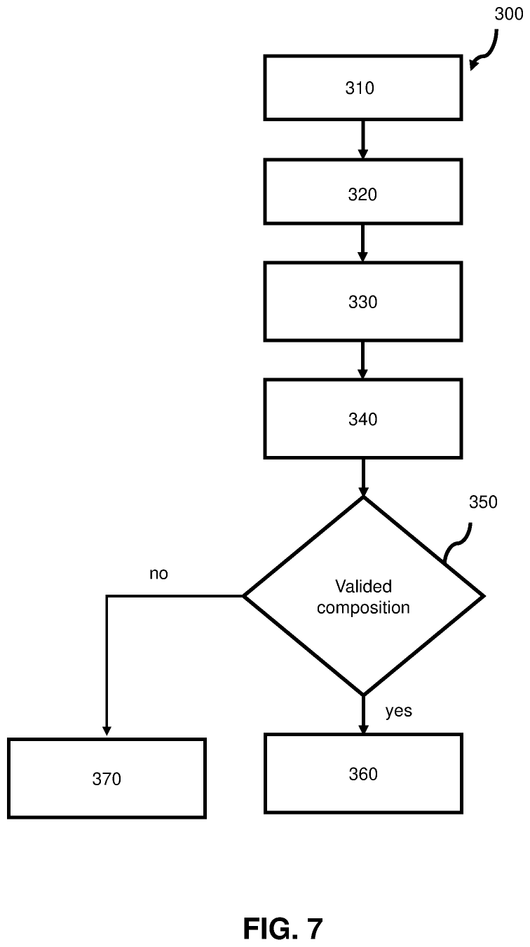

FIG. 7 illustrates a flowchart describing a method 300 for providing manufacturing a sunscreen product.

At block 310, a composition is provided that comprises one or more UV-filter substances with a formulation type. For example, a user may select one or more UV-filter substances, one or more cosmetic ingredients, and a formulation type e.g., via the exemplary interface shown in FIGS. 4A to 4C. Examples of the UV-filter substances may be found in the Annex VI of the Regulation “EC” No. 1223/2009 of the European Parliament and of the Council. Examples of the formulation type may comprise, but are not limited to, oil-in-water fluid emulsion, oil-in-water cream emulsion, water-in-oil emulsion, oil-in-water-in-oil emulsion, water-in-oil-in-water emulsion, water-in-silicone emulsion, silicone-in-water emulsion, polymeric gel cream, lipophilic monophase oil, lipophilic monophase gel, lipophilic monophase stick, lipophilic-alcoholic mixture, hydrophilic monophase fluid, hydrophilic monophase gel, and powder.

At block 320, a calculated performance index of a desired sunscreen product is determined using the provided composition according to the method as described herein. An exemplary method is described with respect to FIG. 3. The user may change e.g., type and/or number of UV-filter substance(s), type and/or number of cosmetic ingredient(s), type of formulation type (e.g., oil-in-water cream, oil-in-water fluid, etc.) e.g., via the exemplary interface shown in FIGS. 4A to 4C until the calculated performance index satisfies a predefined criterion. In some examples, the predefined criterion may be that the difference between the measured performance index and a calculated performance index of the desired sunscreen product is less than a predetermined threshold. In some examples, the predefined criterion may be that the measured performance index is closest to the calculated performance index of the desired sunscreen product.

At block 330, a sunscreen product is manufactured using the composition comprising the one or more UV-filter substances with the formulation type.

At block 340, a measured performance index of the manufactured sunscreen product is provided. The measured performance index may comprise one or more properties, such as SPF and UVA-PF.

At block 350, the measured performance index of the manufactured sunscreen product is compared with the calculated performance index of the desired sunscreen product to determine if the manufactured sunscreen product fulfils predetermined quality criteria.

The comparison may performed by comparing one or more physical, chemical or physio-chemical characteristic(s) that relate to the performance index. For example, the measured SPF of the manufactured sunscreen product may be compared to the calculated SPF of the desired sunscreen product. For example, the measured UVA-PF of the manufactured sunscreen product may be compared to the calculated UVA-PF of the desired sunscreen product.

The calculated performance index of the desired sunscreen product and the measured performance index of the manufactured sunscreen product or any corresponding values derived therefrom may be used for validation. Such validation may be performed by comparing values or value ranges.

If the values lie within an acceptable range or value, such as a 1- or 2-standard deviation(s) interval, the manufactured composition as measured may be valid in the sense that it fulfils the performance criterium or criteria. If the values do not lie within an acceptable range, such as a 1- or 2-standard deviation(s) interval, the manufactured composition as measured may be invalid in the sense that it does not fulfil the performance criterium or criteria.

If the manufactured composition is valid, e.g. a control signal for a production process may be triggered at block 360. Such control signal may be associated with the composition of the produced product. It may control dosing equipment for dosing of different substances of the produced product in the production process.

If the manufactured product is invalid, e.g. a warning signal for the operator of the production process may be triggered at block 370. Such warning signal may signify the invalidity of the manufactured product. The invalidity may trigger a stop signal for the production process. In such cases, an optimization signal may be generated for the production of the produced product to achieve the calculated performance index of the manufactured product. The optimization signal may be used to update the composition which is provided at block 310. For example, the optimization signal may change one or more of: type and/or number of UV-filter substance(s), type and/or number of cosmetic ingredient(s), type of formulation type (e.g., oil-in-water cream, oil-in-water fluid, etc.), and percentages thereof, to achieve a desired performance index of the product to be manufactured. In some examples, the method 300 may be implemented over multiple iterations during trial runs and one or more parameters of the composition may be adjusted for each iteration to approximate the desired performance index. Through sequential iterations, one or more parameters of the composition, such as type and/or number of UV-filter substance(s), type and/or number of cosmetic ingredient(s), type of formulation type (e.g., oil-in-water cream, oil-in-water fluid, etc.), and percentages thereof, may be improved such that the manufactured product fulfils the predetermined quality criteria. If it is determined that the manufactured product is valid, then processing may proceed to block 360, and a production process may start.

By considering the formulation type in determining the sun protection performance, it is possible to reduce the difference between the calculated sun protection performance and the measured sun protection performance in vivo, thereby reducing the number of iterations performed for iteratively improving one or more parameters of the composition with respect to a desired performance index. This may thus reduce extensive trial runs in manufacturing a desired sunscreen product.

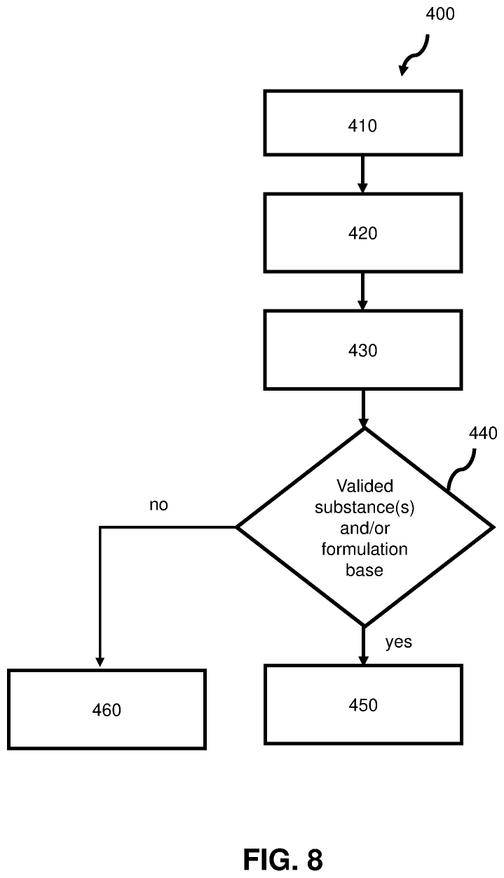

FIG. 8 shows an example of a flowchart describing a method 400 for validating the manufacture of a sunscreen product.

At block 410, a composition is provided that comprises one or more UV-filter substances with a formulation type. The provided composition differs from the composition of an existing produced sunscreen product by at least one different substance and/or is with a different formulation type. The exchange of a substance can be wished for different reasons such as existence of competitive intellectual property rights, regulatory issues in different countries, or lack of resources. The provided composition and the existing produced sunscreen product may comprise different formulation types. For example, the existing produced sunscreen product may be an oil-in-water cream, while the provided composition may be used with an oil-in-water fluid.

At block 420, a calculated performance index of the provided composition according to the method as described herein. An exemplary implementation of the method is described with respect to FIG. 3.

At block 430, a sunscreen product is manufactured using the provided composition that comprises the one or more UV filter substances with the formulation type. In other words, the UV-filter composition is intended to be formulated in the type of formulation (e.g., water-in-oil emulsion). The manufactured product differs from the existing produced sunscreen product by the at least one different substance and/or the different formulation type.

At block 440, a measured performance index of manufactured product and the existing performance index of the existing sunscreen product is compared to validate the at least one substance and/or the different formulation type. If the comparison lies within an acceptable range, the at least one different substance and/or the different formulation type of the manufactured product are valid. On the other hand, if the comparison does not lie within the acceptable range, the at least one different substance and/or the different formulation type are invalid.

If the at least one different substance and/or the different formulation type is valid, e.g. control signal is generated for a production process based on the at least one substance may be triggered at block 450. Such control signal may by be associated with the composition of the sunscreen product including the at least one different substance and/or different formulation type. It may control dosing equipment configured to dose different substances of the sunscreen product in the production process.

If the at least one different substance and/or the different formulation type is invalid, e.g. a warning signal for the operator of the production process may be triggered at block 460. Such warning signal may signify the invalidity of the at least one different substance and/or the different formulation type. This may trigger a stop signal for the production process.

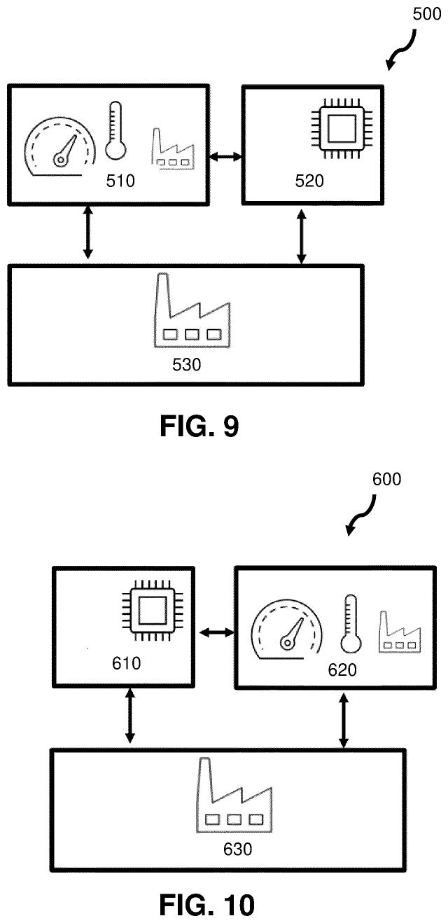

FIG. 9 shows an example of a production line 500 for manufacturing a sunscreen product with a monitoring apparatus 520.

The production line 500 may include dosing equipment 510 configured to dose different substances of the sunscreen product during the production process. The production line 500 may include a conveyor system 530 to convey e.g. bottles, plastic packaging or other suitable packaging to be filled with the sunscreen product. The production line 500 may include a monitoring apparatus 520 configured to monitor quality of the sunscreen product in a production process.

The monitoring apparatus 520 and/or the dosing equipment apparatus 510 may be configured to receive a performance index of the sunscreen product and composition data for the sunscreen product which includes one or more UV-filter substances with a formulation type. The target performance index may include quality criteria like SPF and/or UVA-PF. The monitoring apparatus 520 may be configured to provide the composition data to the dosing equipment. The dosing equipment 510 may be configured to control the dosing based on the provided composition data.

The monitoring apparatus 520 may be configured to receive a measured performance index of the produced product. If the comparison lies within an acceptable range or value, the produced composition fulfils quality criteria. If the comparison does not lie within an acceptable range or value, the produced composition does not fulfil quality criteria. In the latter case, the monitoring unit may be configured to notify an operator or to provide adjusted composition data to the dosing equipment 510.

FIG. 10 shows another example of a production line 600 for manufacturing a sunscreen product with a validation apparatus 610.

The production line 600 may include dosing equipment 620 configured to dose different substances of the sunscreen product in the production process. The production line 600 may include a conveyor system 630 to convey e.g. bottles, plastic packaging or other suitable packaging to be filled with the sunscreen product. The production line 600 may include a validation apparatus 610 configured to validate the production of the sunscreen product.

The validation apparatus 610 may be configured to receive an existing performance index of the sunscreen product (e.g., SPF, UVA-PF, etc.). The validation apparatus 610 may be configured to generate an optimization signal based on the existing performance index. The optimization signal may comprise information about at least one new substance and/or new formulation type. The validation apparatus 610 may be configured to validate the at least one different substance and/or the different formulation type for production of the sunscreen product. The validation apparatus 610 may be configured to compare a performance index of a sunscreen product produced using the new optimization signal and the existing performance index. The validation apparatus 610 may be configured to provide the composition data including the at least one different substance to the dosing equipment.

Combinations and modifications of the embodiments shown in FIGS. 7 and 8 are similarly possible. Both methods exemplify the strength of the methods as described herein. This allows for simplified and more reliable production through monitoring production of the sunscreen product or through validating new substance(s) and/or new formulation type to be used for manufacturing the sunscreen product.

In another exemplary embodiment of the present invention, a computer program or a computer program element is provided that is characterized by being adapted to execute the method steps of the method according to one of the preceding embodiments, on an appropriate system. The computer program element might therefore be stored on a computer unit, which might also be part of an embodiment of the present invention. This computing unit may be adapted to perform or induce a performing of the steps of the method described above. Moreover, it may be adapted to operate the components of the above described apparatus. The computing unit can be adapted to operate automatically and/or to execute the orders of a user. A computer program may be loaded into a working memory of a data processor. The data processor may thus be equipped to carry out the method of the invention.

This exemplary embodiment of the invention covers both, a computer program that right from the beginning uses the invention and a computer program that by means of an up-date turns an existing program into a program that uses the invention.

Further on, the computer program element might be able to provide all necessary steps to fulfil the procedure of an exemplary embodiment of the method as described above.

According to a further exemplary embodiment of the present invention, a computer readable medium, such as a CD-ROM, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

1.-20. (canceled)

21. A computer-implemented method for determining a performance of a sunscreen product comprising at least one UV filter with a formulation type, the method comprising the steps of:
a) providing a computational model to calculate a performance index of a sunscreen product, wherein the computational model applies a continuous film thickness distribution defined by a gamma distribution as a model to determine a film thickness distribution of the applied sunscreen product;
b) providing a desired formulation type of the sunscreen product;
c) adapting the shape parameter c of the gamma distribution to the desired formulation type;
d) determining the performance index of a UV-filter composition for the desired formulation type utilizing the adapted shape parameter of the gamma distribution; and
e) providing the determined performance index of the sunscreen product having the desired formulation type,
wherein the shape parameter c is a function of a total filter concentration, TFC, wherein the function is a saturation like function with a larger slope at a lower TFC and a smaller slope at a higher TFC, and an amplitude of the function is related to a performance of a formulation type in terms of the performance index of the sunscreen product.

22. The computer-implemented method according to claim 21,
wherein the saturation like function comprises:
$$c = a 1 + a 2 \cdot ( \frac{{TFC}^{a 3}}{a 4 + {TFC}^{a 3}} )$$
where c is the shape parameter, a1, a2, a3, and a4 are adjustable parameters, which are determined for each formulation type.

23. The computer-implemented method according to claim 22,
wherein the adjustable parameters, a1, a2, a3, and a4 have the following ranges:
$$0.35 \leq a 1 \leq 0.6 ;$$
$$0.3 \leq a 2 \leq 0 \text{.68} ;$$
$$0.9 \leq a 3 \leq 1.5 ; and$$
$$a 4 \leq 8 .$$

24. The computer-implemented method according to claim 21,
wherein the saturation like function comprises:
$$c = a 1 + a 2 \cdot ( \frac{{TFC}^{a 3}}{a 4 + {TFC}^{a 3}} ) + \frac{a 5}{4} - a 5 \cdot {( REAE - 0.5 )}^{2}$$
where c is the shape parameter, a1, a2, a3, a4, and a5 are adjustable parameters, which are determined for each formulation type, and REAE is a relative erythemally active extinction in the oil-phase.

25. The computer-implemented method according to claim 24,
wherein the parameters, a1, a2, a3, a4, and a5 have the following ranges:
$$0.35 \leq a 1 \leq 0.6 ;$$
$$0.3 \leq a 2 \leq 0 \text{.68} ;$$
$$0.9 \leq a 3 \leq 1.5 ;$$
$$a 4 \leq 8 ; and$$
$$0 \leq a 5 \leq 1 .$$

26. The computer-implemented method according to claim 21,
wherein the saturation like function comprises:
$$c = a 1 + a 2 \cdot ( 1 - {e}^{( - TFC \cdot a 6 )} )$$
where c is the shape parameter, a1, a2, and a6 are adjustable parameters, which are determined for each formulation type, and the parameters a1, a2, and a6 have the following ranges:
$$0.35 \leq a 1 \leq 0.6 ;$$
$$0.3 \leq a 2 \leq 0 \text{.68} ;$$
$$0.1 \leq a 6 \leq 0.9 .$$

27. The computer-implemented method according to claim 21,
wherein the saturation like function comprises:
$$c = a 1 + a 2 \cdot ( 1 - {e}^{( - T FC \cdot a 6 )} ) + \frac{a 5}{4} - a 5 \cdot {( REAE - 0.5 )}^{2}$$
where c is the shape parameter, parameter a5 has a range of 0≤a5≤1, and a1, a2, and a6 are adjustable parameters, which are determined for each formulation type, and the parameters a1, a2, and a6 have the following ranges:
$$0.35 \leq a 1 \leq 0.6 ;$$
$$0.3 \leq a 2 \leq 0 \text{.68} ;$$
$$0.1 \leq a 6 \leq 0.9 .$$

28. The computer-implemented method according to claim 21,
wherein the performance index includes sun protection factor, SPF, and/or UVA protector factor, UVA-PF.

29. The computer-implemented method according to claim 21,
wherein the formulation type comprises one or more of:
Oil-in-water fluid emulsion;
Oil-in-water cream emulsion;
Water-in-oil emulsion;
Oil-in-water-in-oil emulsion;
Water-in-oil-in-water emulsion;
Water-in-silicone emulsion;
Silicone-in-water emulsion;
Polymeric gel cream;
Lipophilic monophase oil;
Lipophilic monophase gel;
Lipophilic monophase stick;
Lipophilic-alcoholic mixture;
Hydrophilic monophase fluid;
Hydrophilic monophase gel; and
powder.

30. The computer-implemented method according to claim 21,
wherein the adjustable parameters of the function determining the adapted shape parameter c are derived from experimental data of SPF and/or UVA-PF.

31. The computer-implemented method according to claim 21,
wherein the sunscreen product further comprises one or more cosmetic ingredients.

32. A method for manufacturing a sunscreen product, the method comprising the steps of:
providing (310) a composition comprising one or more UV-filter substances with a formulation type;
determining a calculated performance index of a desired sunscreen product according to the method of claim 21; and
manufacturing a sunscreen product using the composition comprising one or more UV filter substances within the formulation type.

33. The method according to claim 32, further comprising:
providing a measured performance index of the manufactured sunscreen product; and
comparing the measured performance index of the manufactured sunscreen product with the calculated performance index of the desired sunscreen product to determine if the manufactured sunscreen product fulfils predetermined quality criteria.

34. The method according to claim 32, further comprising:
in response to determining that the manufactured sunscreen product fulfils the predetermined quality criteria, generating a control signal usable for controlling a production process; or
in response to determining that the manufactured sunscreen product does not fulfil the predetermined quality criteria, generating a warning signal usable to signify invalidity of the manufactured product and/or generating an optimization signal usable to change the provided composition such that the manufactured sunscreen product fulfils the predetermined quality criteria.

35. A method for validating the manufacture of a sunscreen product, the method comprising the steps of:
providing a composition comprising one or more UV-filter substances with a formulation type, wherein the provided composition differs from the composition of an existing produced sunscreen product by at least one different substance and/or different formulation type;
determining a calculated performance index of the provided composition according to the method of claim 21;
manufacturing a sunscreen product using the composition comprising one or more UV filter substances within the formulation type, wherein the manufactured product differs from the existing produced sunscreen product by the at least one different substance and/or the different formulation type; and
comparing a measured performance index of manufactured product and the existing performance index of the existing sunscreen product to validate the at least one substance and/or the different formulation type.

36. An apparatus for determining a composition comprising one or more UV-filter substances with a formulation type, the apparatus comprising one or more processing units configured to determine a composition of filter substances to form the sunscreen composition, wherein the one or more processing units include instructions, which when executed on the one or more processing units execute the method steps of claim 21.

37. An apparatus for manufacturing a sunscreen product, the apparatus comprising:
a monitoring apparatus; and
a dosing equipment;
wherein the monitoring apparatus is configured to control the dosing equipment to manufacture the sunscreen product according to claim 32.

38. An apparatus for validating manufacture of a sunscreen product, the apparatus comprising one or more processing unit(s) configured to validate production of a sunscreen product, wherein the processing unit(s) include instructions, which when executed on the one or more processing unit(s) preform the method of claim 35.

39. A computer program element comprising instructions, which when executed by a processing unit, cause the processing unit to carry out the steps of the method of claim 21.

40. A computer-readable medium having stored thereon the computer program element of claim 39.
